(19) United States
(12) Patent Application Publication
(10) Pub. No.: US 2021/0139863 A1
(43) Pub. Date: May 13, 2021

(54) CONJUGATES OF FATTY ACID-THERAPEUTIC PROTEINS FOR HALF-LIFE EXTENSION AND USE OF THE SAME

(71) Applicant: GWANGJU INSTITUTE OF SCIENCE AND TECHNOLOGY, Gwangju (KR)

(72) Inventors: Inchan KWON, Gwangju (KR); Jinhwan CHO, Gwangju (KR); Junyong PARK, Gwangju (KR)

(21) Appl. No.: 17/094,994

(22) Filed: Nov. 11, 2020

(30) Foreign Application Priority Data
Nov. 11, 2019 (KR) 10-2019-0143851

(51) Int. Cl. C12N 9/06; A61K 47/60

(52) U.S. Cl. CPC C12N 9/0048; C12Y 107/03003; A61K 47/60

(57) ABSTRACT

The present invention relates to a conjugate capable of controlling in vivo half-life, which comprises urate oxidase; a pharmaceutical composition with increased in vivo half-life for preventing or treating gout, which comprises the conjugate or a pharmaceutically acceptable salt thereof; and a method for preventing or treating gout using the same.

It was found that the conjugate of the present invention has a very important role in the extension of in vivo serum half-life by controlling its binding competition with a neonatal Fc receptor (FcRn) for serum albumin (SA) based on the length of its linker, and this finding has significance with respect to its application as an agent for gout treatment and extension of application of fatty acid (FA) conjugation to therapeutic proteins having a high molecular weight.

### CROSS-REFERENCE TO RELATED APPLICATION

This application claims priority under 35 U.S.C. § 119 to Korean Patent Application No. 10-2019-0143851, filed on Nov. 11, 2019 in the Korean Intellectual Property Office (KIPO), the contents of which are herein incorporated by reference in their entirety.

### TECHNICAL FIELD

The present invention relates to a conjugate capable of controlling in vivo half-life, which comprises urate oxidase; a pharmaceutical composition with increased in vivo half-life for preventing or treating gout, which comprises the conjugate or a pharmaceutically acceptable salt thereof; and a method for preventing or treating gout using the same.

### BACKGROUND ART

The global market for therapeutic proteins is growing rapidly. The market was worth USD 10.8 billion in 2010 and is expected to reach USD 29.8 billion by 2020. Although therapeutic proteins have excellent functions (e.g., excellent effects and biological safety), one of the main problems that have arisen in the development of therapeutic proteins is their short in vivo half-life caused by rapid purification from blood circulation due to intracellular degradation, proteolysis, kidney filtration, etc. Therefore, it is important to develop long-acting therapeutic proteins so as to reduce the inconvenience which is caused by repeated administration and the cost of treatment.

The conjugation of polyethylene glycol (PEG) has been used to extend the circulation half-life of therapeutic proteins, but it has several problems (e.g., immunogenicity, non-degradability, etc.), and thus alternative methods are needed.

As an alternative to PEG, human serum albumin (HSA) is of great interest due to its low immunogenicity, good biocompatibility/degradability, and very long serum half-life (3 weeks or longer). The long serum half-life of HSA in the human body is achieved by avoiding intracellular degradation through FcRn-mediated recycling and reduced filtration in the kidneys. Therefore, gene fusion or covalent conjugation to HSA has been used to extend the serum half-life of therapeutic peptides/proteins.

However, the gene fusion and the covalent conjugation to HSA have several problems, including reduced expression levels, low conjugation yields, complicated processes, and high costs.

Conjugation of a fatty acid (hereinafter, FA); and a serum albumin (hereinafter, SA) ligand to therapeutic peptides/proteins has been studied with respect to half-life extension using a non-covalent albumin binding in vivo. FA conjugation has advantages over direct conjugation by HSA in that FA provides a higher conjugation/production yield, lower production cost, deep penetration into tissue, higher activity-to-mass ratio, etc., thus making the development of long-acting therapeutic peptides/proteins more promising.

Thus far, the FA conjugations to some therapeutic peptides/proteins including glucagon-like peptide-1 (3.3 kDa), exendin-4 (4.2 kDa), insulin (5.9 kDa), a human growth factor (22 kDa), and interferon-a2 (25 kDa) have successfully extended serum half-life in vivo. All of these are therapeutic peptides and small proteins (up to 25 kDa).

Surprisingly, no reports have been released with respect to serum half-life extension through FA conjugation to a therapeutic protein having a molecular weight of greater than 25 kDa. In the case of urate oxidase (hereinafter, Uox), which is a large therapeutic protein (135 kDa) for the treatment of gout, FA conjugation did not substantially increase serum half-life in vivo. Therefore, the present inventors have assumed that an increase in serum half-life through FA conjugation may be dependent on protein size and may not be effective for therapeutic proteins with a high molecular weight.

Extension of half-life is an important issue to solve even for large therapeutic proteins. One of the underlying reasons is that according to protein size distribution analysis, most human proteins that are potential therapeutic targets appear to have a molecular weight of greater than 25 kDa.

Another reason is that most therapeutic proteins, which were recently (2011 to 2016) approved by the U.S. Food and Drug Administration (FDA) (e.g., asparaginase (140 kDa), a Cl esterase inhibitor (105 kDa), and a von Willebrand factor (280 kDa)), have a high molecular weight. During the same period, long-acting versions of therapeutic proteins having a high molecular weight, including a vascular endothelial growth factor receptor (151 kDa) and factor VIII (166 kDa), were approved by the FDA. Therefore, considering the advantages in the half-life extension technology, it is worthwhile to review the expansion of the application of FA conjugation to therapeutic proteins having a high molecular weight.

In the case of large proteins (e.g., Uox, etc.), the limited extension of half-life in vivo through FA conjugation may be due to ineffective FcRn-mediated recycling. In particular, considering the bulkiness of therapeutic proteins, it is possible that FA-conjugated therapeutic proteins may compete with FcRn binding to SA, which is protein size-dependent. The present inventors have assumed that the sizes of therapeutic proteins and small proteins were too small to compete with FcRn binding to SA (FIG. 1B). However, as the size of the protein increases, the competition with FcRn binding to SA will increase (FIG. 1C). To confirm this hypothesis, palmitic acid (PA) and Uox were selected as models of an FA and a therapeutic protein having a high molecular weight, respectively.

FAs with a longer aliphatic chain have albumin binding affinity that is stronger than a normal level of binding affinity, but these FAs show a higher hydrophobicity and a lower solubility in water, thus complicating the conjugation process.

Palmitic acid (hereinafter, PA) is the most common fatty acid (FA) in the human body, and it has a physiologically important role as well as a long aliphatic chain sufficient for efficient binding to albumin.

Since FAs have low solubility to water, many researchers have attempted to modify them to increase their solubility. However, the present inventors have already measured the conditions to increase their solubility by using solubilizers and sodium deoxycholate (DCA).

Uox, having a high molecular weight, is suitable for studying the competition with FcRn binding to SA. Additionally, the conjugation of PA to Uox did not substantially increase serum half-life in vivo. Assuming that there is a strong competition of PA-conjugated Uox (Uox-PA) with FcRn binding to SA, one way to reduce this competition is to extend the distance between large proteins and FcRn.

Conventionally, the carboxyl group of FA binds to the amine group of therapeutic peptides/proteins and thereby generates a very short linker. Even for FA-conjugated Uox, FA is directly bound to the amine group of Uox. Since such a short linker can induce a short distance between the protein and SA, it can induce competition against FcRn binding to SA.

Therefore, the present inventors assumed that a longer linker between PA and Uox could increase the distance between Uox-PA and FcRn (FIG. 1D). Although several relatively long linkers were reported, based on the knowledge of the present inventors, the correlation between the size of therapeutic proteins and the increase of serum half-life, or the correlation between the length of an FA linker and the increase of serum half-life has not been reported.

Accordingly, the present inventors have confirmed that the use of an FA linker longer than a critical length, by controlling the length of the linker between the large therapeutic proteins (e.g.,Uox, etc.) and FA, can substantially reduce the competition of Uox-PA with FcRn binding to SA and provide increased serum half-life in vivo, thereby completing the present invention.

### PRIOR ART DOCUMENTS

### Patent Documents

Patent Document 1. Korean Patent No. 10-1637010 B1

### DISCLOSURE

### Technical Problem

An object of the present invention is to provide a conjugate capable of controlling half-life in vivo, containing urate oxidase.

Another object of the present invention is to provide a pharmaceutical composition with increased in vivo half-life for preventing or treating gout, which contains the conjugate or a pharmaceutically acceptable salt thereof.

Still another object of the present invention is to provide a method for preventing or treating gout, which includes a step of administering the conjugate or a pharmaceutically acceptable salt thereof to a subject excluding humans.

### Technical Solution

The present invention is described in detail as follows. Meanwhile, respective descriptions and embodiments disclosed in the present invention may also be applied to other descriptions and embodiments. That is, all combinations of various elements disclosed in the present invention fall within the scope of the present invention. Further, the scope of the present invention cannot be considered to be limited by the specific description below.

To achieve the above objects, an aspect of the present invention provides a conjugate capable of controlling half-life in vivo, containing urate oxidase.

The conjugate may be a conjugate having the following General Formula 1.

[General Formula 1]

[chemical structure]

wherein in General Formula 1 above,

Uox is urate oxidase;

X is a polymer;

Y is a fatty acid; and

half-life in vivo can be controlled according to the length from Uox to Y.

As used herein, the term “conjugate” or “conjugate of General Formula 1” may refer to a compound in which the binding between urate oxidase (Uox) and X-Y is connected by an amine group as in General Formula 1 above, and it has a characteristic that the half-life in vivo can be controlled according to the length from Uox to Y. The term “conjugate” can be used interchangeably with the term “Uox-PA conjugate”.

In particular, “the length from Uox to Y” may refer to a distance between the ε-carbon in a lysine residue of Uox and a carbonyl carbon of Y.

In addition, the part from Uox to Y may be referred to as a “linker”.

As used herein, the term “linker” may refer to the part from Uox to Y in General Formula 1 above (i.e., the distance between the ε-carbon in a lysine residue of Uox and a carbonyl carbon of Y). In an embodiment, the linker may refer to a part which links Uox and a fatty acid (palmitic acid), but the linker is not limited thereto.

The conjugate has a characteristic that enables increasing the half-life in vivo or maintains it in an increased state compared to when Uox is used alone, by controlling the length from Uox to Y.

Specifically, when the length from Uox to Y is greater than 0.2 nm and equal to or less than 3 nm, the half-life in vivo may increase. In an embodiment, it was confirmed that when the length from Uox to Y was 0.25 nm to 2.8 nm, the half-life was increased by about 2.1-fold to 7.5-fold. In addition, it was confirmed that when the length from Uox to Y was greater than 0.2 nm and equal to or less than 3 nm, the half-life was increased in direct proportion to the increase in its length (FIGS. 4A-4B).

In addition, it was confirmed that when the length from Uox to Y was greater than 3 nm and equal to or less than 5 nm, the increase rate of the half-life in vivo was reduced and maintained for 8 to 10 hours (FIG. 4B).

As used herein, the term “urate oxidase (Uox)”, which is a large therapeutic protein (135 kDa) for treating gout, refers to an enzyme that oxidizes uric acid to allantoin. Allantoin is 5 to 10 times more soluble than uric acid, thus making it easy to excrete into the kidneys. Allantoin is normally present in mammals, but it is deficient in primates (e.g., humans) due to a nonsense mutation. At present, uricozyme extracted from Aspergillus fluvus or rasburicase (i.e., a recombinant uricase) is used for hyperuricemia and tumor lysis syndrome (TLC) associated with malignant tumor.

Although uricozyme and rasburicase have a strong effect of reducing uric acid levels, they have short half-lives and thus can only be used as an injection. Additionally, they have high side effects and high antibody expression rates due to immune responses, and the stability for their long-term use has not been established. Therefore, it is difficult to use uricozyme and rasburicase as therapeutic agents for chronic gout, and studies to reduce the antigenicity of rasburicase while prolonging their half-lives are underway.

The urate oxidase of the present invention can reduce immune responses, and regulate and increase half-life in vivo by forming a conjugate, and thus, it can be effectively used as a therapeutic agent for gout. As used herein, the terms “Uox”, “urate-oxidizing enzyme”, “urate oxidase”, “therapeutic protein”, and “large protein” can be used interchangeably with one another.

The urate oxidase can exist as a tetrameric structure.

In particular, the tetrameric structure may refer to a form of a protein having a quadruple structure consisting of four Uox subunits (monomers).

Additionally, the X of the conjugate of General Formula 1 may be a polymer.

As used herein, the term “polymer” refers to a type of polymer in which units are repeatedly linked. In a specific embodiment, the polymer may include polyethylene glycol (PEG) or dibenzocyclooctyne (DBCO), and PEG or DBCO may be included in a form in which each or a combination thereof is repeatedly linked, but the polymer is not limited thereto.

In a specific embodiment, the polymer may have a structure in which PEG is repeated, and may have a certain size by the structure. More specifically, the polymer may be NHS-PEG₂ₖ, wherein the size of PEG may be 2 kDa.

Additionally, the length of the polymer can be controlled by the number of PEGs, but is not limited thereto.

The polymer may be one which is prepared by a chemical bond between an azide group and a DBCO group, but the preparation method is not limited thereto.

In particular, the azide group may be NHS-Azide or NHS-PEGₙ-azide (wherein n is an integer which is equal to or greater than 0 and equal to or less than 10), and specifically, the azide group may be NHS-PEG₄-azide or NHS-PEG₈-azide, but the azide group is not limited thereto.

The DBCO group may be DBCO-amine or DBCO-PEGₙ-amine (wherein n is an integer which is equal to or greater than 0 and equal to or less than 10), and specifically, the DBCO group may be DBCO-PEG₄-amine, DBCO-PEG₆-amine, DBCO-PEG₈-amine, or DBCO-PEG₉-amine, but the DBCO group is not limited thereto.

The half-life of the urate oxidase in vivo can be increased or maintained in an increased state by controlling the length of the polymer.

Additionally, the Y of the conjugate of General Formula 1 may be a fatty acid.

As used herein, the term “fatty acid (FA)” refers to carboxylic acid, which has an aliphatic chain consisting of an even number of carbon atoms among 4 to 28, which is either saturated or unsaturated. In a specific embodiment, the fatty acid may be a fatty acid including C₁₀₋₂₀, and more specifically, the fatty acid may be palmitic acid (PA), lauric acid, myristic acid, or stearic acid, but the fatty acid is not limited thereto.

The use of palmitic acid as the fatty acid has an advantage in that Uox and palmitic acid will have a homo-tetrameric structure, thus enabling the binding with a plurality of palmitic acid units.

In an embodiment of the present invention, as a result of mass spectrum analysis of Uox-palmitic acid conjugates according to the length of each linker, it was confirmed that the number of palmitic acid units conjugated to a single molecule of Uox (i.e., a homo-tetramer) was 6 to 10 (FIG. 11).

The conjugate of the present invention may be any one or more selected from Formulas 1 to 4 below.

[chemical structure]

Additionally, the conjugate can form a complex with serum albumin (SA) and a neonatal Fc receptor (FcRn) in vivo.

The complex may be in the form of a tertiary structure of FcRn/SA/Uox-PA, but the structure of the complex is not limited thereto (FIGS. 1A-1D).

The Uox-palmitic acid conjugate can form a primary complex by binding with serum albumin in vivo, and the increase of the half-life of the conjugate can be induced through FcRn-mediated recycling by binding with FcRn present in vivo (FIGS. 1A-1D).

In an embodiment of the present invention, it was confirmed that when the Uox-palmitic acid conjugate had no linker or the length of the linker was as short as 0.2 nm or less, the FcRn/SA/Uox-PA complex of the tertiary structure was not generated. Specifically, it was confirmed that the FcRn/SA/Uox-PA complex could be generated when the length of the linker was 0.25 nm (i.e., UP01 of FIGS. 5A-5D) or longer.

Additionally, the complex formed between UP01-04 and FcRn and SA was much larger compared to when an unmodified Uox was used, from which it could be predicted that it is highly likely that this causes the extension of serum half-life through FcRn-mediated recycling. However, it can be seen that only a small fraction of UP01 was involved in the formation of the tertiary structure (FcRn/SA/UP01), which indicates competition with FcRn binding to SA.

Additionally, in an embodiment of the present invention, it was confirmed that an increase in the distance between PA and Uox in the Uox-PA conjugate induces a substantial increase of serum half-life in vivo, which suggests it is highly likely that this causes the reduction in competition with FcRn binding to SA.

Specifically, it was confirmed that when a Uox-PA conjugate with a short linker is attached to SA, the Uox-PA conjugate collides with FcRn (FIG. 6B), and in addition, it was confirmed that when the length of the linker was increased to 1.5 nm, the Uox-PA conjugate did not come into contact with SA, and thus the collision disappeared (FIG. 6C). Additionally, it was confirmed that when the length of the linker was 2.5 nm, the intramolecular distance between Uox and SA was such that they were far off from each other, and thus no collision occurred between them (FIG. 6D).

That is, in the case of a Uox-PA conjugate, it was confirmed through a specific embodiment that there is a strong correlation between serum half-life extension and tertiary structure formation of FcRn/SA/Uox-PA, from which it was confirmed that FcRn-mediated recycling is a major mechanism for extending the half-life of a Uox-PA conjugate in vivo.

Additionally, it was confirmed that the tertiary complex formation increased as the linker length increased, which indicates that the increase in the linker length of the conjugate reduced the competition of the conjugate with FcRn binding to SA and extended its half-life in vivo.

From the above, it was confirmed that the Uox-palmitic acid conjugate can form a tertiary structure of an FcRn/SA/Uox-PA complex by controlling the linker length, and that the half-life of the conjugate can be increased by forming the above complex.

Another aspect of the present invention provides a pharmaceutical composition with increased in vivo half-life for preventing or treating gout, which contains the conjugate or a pharmaceutically acceptable salt thereof.

In the present invention, it was confirmed that the in vivo half-life can be increased using the conjugate containing Uox as one constitution and thus could be effectively used in the prevention or treatment of gout.

As used herein, the term “gout” refers to a form of arthritis that occurs due to monosodium urate crystals (MSUs) produced by hyperuricemia. It is known that excluding the decrease in renal excretory function, the remaining 10% to 15% of hyperuricemia is caused by overproduction of uric acid, the causes of which are genetic defects in the process of purine metabolism, problems in the process of ATP metabolism, diseases that increase the rate of cell conversion, etc.

As used herein, the term “treatment” refers to all actions that inhibit or delay the onset of gout by the administration of a pharmaceutical composition containing the above conjugate or a pharmaceutically acceptable salt thereof.

As used herein, the term “prevention” refers to all actions that inhibit or beneficially change the symptoms of gout by the administration of a pharmaceutical composition containing the above conjugate or a pharmaceutically acceptable salt thereof.

In particular, the term conjugate is the same as above.

The pharmaceutical composition of the present invention may further contain a pharmaceutically acceptable carrier, excipient, or diluent, which is commonly used in the preparation of pharmaceutical compositions. The carrier may contain a carrier which is not naturally occurring.

Specific examples of the carrier, excipient, or diluent may include lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate, mineral oil, etc., but the carrier, excipient, or diluent is not limited thereto.

Additionally, the pharmaceutical composition may have any one formulation selected, according to the conventional method, from the group consisting of tablets, pills, powders, granules, capsules, suspensions, solutions for internal use, emulsions, syrups, sterile aqueous solutions, non-aqueous solvents, lyophilized preparations, and suppositories, and the pharmaceutical composition may be in various oral or parenteral formulations. The formulations are prepared using diluents or excipients (e.g., fillers, extenders, binders, humectants, disintegrants, surfactants, etc.) that are commonly used. Solid formulations for oral administration may include tablets, pills, powders, granules, capsules, etc. The solid formulations may be prepared using at least one excipient (e.g., starch, calcium carbonate, sucrose, lactose, gelatin, etc.). Moreover, in addition to the simple excipients, lubricants (e.g., magnesium stearate, talc, etc.) may also be used. Liquid formulations for oral administration may include suspensions, solutions for internal use, emulsions, syrups, etc. In addition to simple diluents commonly used (e.g., water and liquid paraffin), various excipients (e.g., humectants, sweeteners, fragrances, preservatives, etc.) may also be used. Formulations for parenteral administration may include sterile aqueous solutions, non-aqueous solvents, suspensions, emulsions, lyophilized preparations, suppositories, etc. The non-aqueous solvents and the suspensions may include propylene glycol, polyethylene glycol, vegetable oil (e.g., olive oil), an injectable ester (e.g., ethyloleate), etc. A base for the suppositories may include witepsol, macrogol, tween 61, cacao butter, laurin butter, glycerogelatin, etc., but is not limited thereto.

In an embodiment of the present invention, the half-life of the Uox-PA conjugate was measured in vivo so as to confirm the correlation between half-life extension and competition with FcRn binding to SA in mice. SA binding and formation of a tertiary complex of FcRn/SA/Uox-PA were confirmed using mouse serum albumin (MSA) and mouse FcRn. In addition, it was confirmed that the binding of a Uox-PA conjugate and the tendency of formation of the tertiary complex of FcRn/SA/Uox-PA to HSA were very similar to that to mouse serum albumin (MSA), and the results could be also confirmed in HSA having identity to MSA by 85%.

Therefore, the above results suggest that the pharmaceutical composition of the present invention, which contains the conjugate with increased half-life, can be effectively used for the prevention and treatment of gout.

Still another aspect of the present invention provides a method for preventing or treating gout, which includes a step of administering the above conjugate or a pharmaceutically acceptable salt thereof to a subject excluding humans.

In particular, the conjugate, prevention, and treatment are the same as described above.

As used herein, the term “administration” refers to the introduction of the pharmaceutical composition to a subject by any appropriate manner.

As used herein, the term “subject” refers to all animals including humans, rats, mice, cattle, etc., in which gout has occurred or can occur. The animal may be a mammal including not only humans but also cattle, horses, sheep, pigs, goats, camels, antelopes, dogs, cats, etc. in need of treating symptoms similar to gout, but the animal is not limited thereto.

The pharmaceutical composition of the present invention may be administered in a pharmaceutically effective amount.

The term “pharmaceutically effective amount” refers to an amount sufficient to treat diseases at a reasonable benefit/risk ratio applicable to any medical treatment. The effective dose can be determined according to factors which include the type of a subject and severity, age, sex, drug activity, sensitivity to drug, administration time, administration route and excretion rate, duration of treatment, and other drugs used simultaneously, and other factors well known in the medical field.

The pharmaceutical composition may be administered as an individual therapeutic agent or in combination with other therapeutic agents, and it may be administered sequentially or simultaneously with conventional therapeutic agents. Additionally, the pharmaceutical composition may be administered once or multiple times. Considering all of the above factors, it is important to administer an amount that can achieve the maximum effect in a minimal amount without side effects, and this can easily be determined by those skilled in the art.

Additionally, the pharmaceutical composition may be administered orally or parenterally (e.g., intravenously, subcutaneously, intraperitoneally, or topically applied) according to the desired method. The administration dose may vary depending on the patient's conditions and body weight, severity of disease, drug forms, and the route and time of administration, but it may be appropriately selected by those skilled in the art. In a specific embodiment, the pharmaceutical composition may be generally administered once or in several divided doses daily, and a preferred dose may be appropriately selected by those skilled in the art according to the conditions and weight of a subject, severity of disease, drug forms, and the route and duration of administration.

### Advantageous Effects

The present inventors have confirmed that the linker length of the conjugate of the present invention can have a very important role in extending serum half-life in vivo by controlling the competition of the conjugate with FcRn binding to serum albumin (SA). Therefore, the significance of the present invention lies in the application of the conjugate as a therapeutic agent for gout, and the expansion of application of a fatty acid (FA) conjugation to therapeutic proteins having a high molecular weight.

### BRIEF DESCRIPTION OF DRAWINGS

FIGS. 1A-1D show schematic diagrams illustrating FcRn-mediated recycling of FA-mediated therapeutic proteins, and tertiary complexes of FcRn/SA/FA-conjugated proteins with various sizes and linker lengths.

FIG. 1A shows a schematic diagram illustrating that the FA binds to serum albumin (SA) when the FA-mediated therapeutic protein (therapeutic protein-FA) is injected into the blood. The FA-conjugated therapeutic protein forms a complex with SA and binds to FcRn in endosomes under acidic conditions, while serum proteins do not. Therefore, the FA-mediated therapeutic protein avoids lysosomal degradation through FcRn-mediated recycling of SA and thereby extends the half-life in vivo.

FIG. 1B shows a schematic diagram illustrating that an FA-conjugated small protein, which forms a complex with SA by a short linker, does not compete with the binding of FcRn to SA.

FIG. 1C shows a schematic diagram illustrating that when a large protein is conjugated to the FA by a short linker, the FA-conjugated large protein, which forms a complex with SA, competes with the binding of FcRn to SA.

FIG. 1D shows a schematic diagram illustrating that a large protein conjugated to an FA by a long linker allows the binding of FcRn to SA.

FIGS. 2A-2B show drawings illustrating the structures and characteristics of Uox-PA conjugates with various linker lengths.

FIG. 2A shows a drawing illustrating tetrameric Uox, which is represented by four circles, and Uox-PA conjugates, each of which being linked by a linker, are each represented by a symbol. The length of each linker was measured using Chem3D, and each linker is indicated with an arrow. The length of each linker was obtained by measuring the distance between the ε-carbon in a lysine residue of Uox and a carbonyl carbon of PA when the linker was maximally stretched. In the case of UP01, a gap of 0.25 nm was inevitably generated using NHS-PA, and in the case of UP02, a linker length was added without PEG through a SPAAC reaction compared to UP01. The linker length of UP03 was increased by 4 repeats of PEG in the linker length of UP02, and the linker length of UP04 was increased by 8 repeats of PEG in the linker length of UP02.

FIG. 2B shows protein gel images of purified Uox and Uox-PA conjugates. Uox-PA conjugates have greater molecular weights than Uox due to various linker lengths. The images were obtained using Bio-Rad ChemiDoc™ XRS⁺ (Lane M, molecular weight markers; Lane 1, Uox; Lane 2, UP01; Lane 3, UP02; Lane 4, UP03; and Lane 5, UP04).

FIG. 3A-3B shows graphs which illustrate the binding of Uox-PA conjugates to SA and determination of the half-maximal binding concentration (BC₅₀). All of the experiments were performed at pH 7.4. The affinity of each Uox-PA conjugate bound to SA was measured using 6× His tag ELISA. In both FIG. 3A (MSA) and FIG. 3B (HSA), the Uox-PA conjugates were each spread on a plate, subjected to Uox-PA with different concentrations, and incubated, and the remaining amount of each Uox-PA conjugate was measured by ELISA. Each point in each graph represents the mean±SD (standard deviations) (n=3). The graphs were fitted by nonlinear regression in OriginPro. The BC₅₀ was calculated according to the manufacturer's manual.

FIGS. 4A-4B show graphs which illustrate the measurement results of serum half-lives of Uox-PA conjugates in mice after a single intravenous injection.

FIG. 4A shows a graph which illustrates the measurement results of serum activities of Uox and Uox-PA conjugates in mice plasma at each time point after intravenous administration. Each point in the graph data represents the mean±SD (n=5). The serum activity on a logarithmic scale over time was plotted so as to provide a linear fit. The serum half-lives of Uox and Uox-PA conjugates can be confirmed in the table.

FIG. 4B shows a graph which illustrates a correlation between the serum half-life and the linker length. The serum half-life continued to increase until the linker length became 2.8 nm. When the length was increased to 2.8 nm or longer, the serum half-life appeared to be saturated.

FIGS. 5A-5D show graphs illustrating experimental results with respect to formation of FcRn/SA/Uox-PA tertiary complexes. All of the experiments were performed at pH 6.0.

FIG. 5A shows a graph which illustrates the amount of Uox or Uox-PA conjugates bound in vitro on MSA, which is not bound to mouse FcRn; FIG. 5B shows a graph which illustrates the amount of Uox or Uox-PA conjugates bound in vitro on MSA, which is bound to mouse FcRn; FIG. 5C shows a graph which illustrates the amount of Uox or Uox-PA conjugates bound in vitro on HSA, which is not bound to human FcRn; and FIG. 5D shows a graph which illustrates the amount of Uox or Uox-PA conjugates bound in vitro on HSA, which is bound to human FcRn. The amount of Uox or Uox-PA conjugates was measured by ELISA and normalized as relative binding affinity to the highest signal obtained. The graph represents the mean±SD (n=3). *P<0.01; N.S.: not significant (two-tailed student t-test).

FIGS. 6A-6C show drawings illustrating the prediction of tertiary complex formation of an FcRn/SA/FA-conjugated protein according to the size of a protein and the linker length of the FA.

FIG. 6A shows a drawing illustrating the prediction of formation of an FA-conjugated tertiary complex of FcRn/SA in Uox with a 0.24 nm linker; FIG. 6B shows a drawing illustrating the prediction of formation of an FA-conjugated tertiary complex of FcRn/SA in Uox with a 1.5 nm linker; and FIG. 6C shows a drawing illustrating the prediction of formation of an FA)-conjugated tertiary complex of FcRn/SA in Uox with a 2.8 nm linker. The linkers are marked with a wavy line. The structures were generated by PyMOL (www.pymol.org) using PDB files (ID: 1ITF, 1WS2, and 4N0F).

FIG. 7 shows a drawing illustrating the structure of Uox. The Uox was based on the PDB file (ID: 1WS2). The diameter is marked with an arrow. The structure was indicated and the diameter was calculated by PyMOL.

FIG. 8 shows an image illustrating a purified Uox band in a sodium dodecyl sulfate-polyacrylamide gel electrophoresis (SDS-PAGE). The purified Uox was loaded on a 12% polyacrylamide gel. The protein was detected by staining with Coomassie Brilliant Blue and photographed by using Bio-Rad ChemiDoc™ XRS⁺. M represents a lane for the standard of a molecular weight.

FIG. 9 shows drawings relating to chemical structures and reaction schemes. A represents an NHS-amine reaction; B represents a strain-promoted azide-alkyne cycloaddition (SPAAC) reaction; C represents NHS-PA; D represents DBCO-amine; E represents DBCO-PEG₄-amine; F represents DBCO-PA; G represents DBCO-PEG₄-PA; and H represents azidoacetic acid-NHS; and I represents azido-PEG₄-NHS. All of the structures were drawn by ChemDraw.

FIG. 10 shows an entire SDS-PAGE gel image of FIG. 2B. Uox and Uox-PA conjugates were loaded on a 12% polyacrylamide gel as molecular weight standards, and proteins were detected by staining with Coomassie Brilliant Blue. The proteins were detected by staining with Coomassie Brilliant Blue and photographed by using Bio-Rad ChemiDoc™ XRS⁺ (Lane M represents molecular weight markers; Lane 1, Uox; Lane 2, UP01; Lane 3, UP02; Lane 4, UP03; and Lane 5, UP04).

FIG. 11 shows drawings illustrating the results of MALDI-TOF mass spectrometry performed on Uox and Uox-PA conjugates.

(1) shows the result of a mass spectrum of Uox, which indicates a major peak of 34,926 m/z.

(2) shows the result of a mass spectrum of UP01 containing 0.15% DCA, which indicates 4 peaks from 0 palmitic acid conjugation (PA 0) to 3 palmitic acid conjugations (PA 3).

(3) shows the result of a mass spectrum of UP01 containing 0.30% DCA, which indicates 9 peaks from PA 0 to PA 8.

(4) to FIG. 11(6) show the results of mass spectra of UP02, UP03, and UP04. The Table shown in FIG. 11 summarizes the information on the mass spectrum peaks of Uox, UP01 (0.15% DCA), UP01 (0.30% DCA), UP02, UP03, and UP04, and the number of conjugated palmitic acid units.

FIGS. 12A-12B show schematic diagrams illustrating an in vitro binding assay.

FIG. 12A shows a schematic diagram relating to an in vitro SA binding assay. Amine-binding plates were coated with an appropriate amount of SA, incubated with Uox or Uox-PA conjugates, and then washed. The Uox or Uox-PA conjugates remaining in an excess amount appeared to have strong SA binding affinity.

FIG. 12B shows a schematic diagram relating to formation of a tertiary complex of FcRn/SA/Uox-PA assay, which was designed to confirm the interactions between FcRn/SA/Uox-PA. FcRn was spread on amine-binding plates and then allowed to bind to SA. Then, the binding to albumin of Uox-PA conjugates was tested. Although Uox-PA conjugates can bind to SA, if the Uox-PA conjugates compete with FcRn, then FcRn/SA complex will be dissociated and the Uox-PA conjugates will not be detected. The amount of the remaining Uox or Uox-PA conjugates in the plate is considered to have the complete tertiary complex of FcRn/SA/Uox-PA.

FIGS. 13A-13B show graphs illustrating the correlation between BC₅₀ and a linker length. The correlation between BC₅₀ and each linker length of the Uox-PA conjugates with respect to MSA (FIG. 13A) and HSA (FIG. 13B) were weak. The graphs were fitted by linear regression in OriginPro, and the coefficient of determination was calculated.

FIG. 14 shows a graph relating to relative enzymatic activity of Uox and Uox-PA conjugates. The relative enzymatic activities of Uox-PA conjugates were normalized using the enzymatic activity of Uox. Compared to the enzymatic activity of Uox, the enzymatic activity of UP01 was significantly reduced by DCA at higher relative concentrations to meet fatty acid conjugation with a certain yield.

The graph represents the mean±SD (n=3). *P<0.01; N.S.: not significant (two-tailed student t-test).

FIGS. 15A-15B show graphs illustrating the correlation between BC₅₀ and half-life of Uox-PA conjugates. The correlation between BC₅₀ and half-life of the Uox-PA conjugates with respect to MSA (FIG. 15A) and HSA (FIG. 15B) were weak. The graphs were fitted by linear regression in OriginPro, and the coefficient of determination was calculated.

FIGS. 16A-16B show graphs illustrating the correlation between the relative binding affinity of the of Uox-PA and FcRn/SA complex and half-life. The correlation between the relative binding affinity with an FcRn/SA complex for MSA (FIG. 16A) and HSA (FIG. 16B) and the half-life of the Uox-PA conjugates was strong. The graphs were fitted by linear regression in OriginPro, and the coefficient of determination was calculated.

### DETAILED DESCRIPTION OF THE INVENTION

Hereinafter, the present invention will be described in more detail with reference to the following Examples. However, these Examples are for illustrative purposes only and the scope of the invention is not limited by these Examples.

### EXPERIMENTAL EXAMPLE 1

### Cloning, Expression, and Purification of Urate Oxidase (Uox)

For cloning, expression, and purification of Uox, a plasmid encoding the Uox gene was transformed into TOP10 E. coli (Hahn, I. Kwon, Generation of therapeutic protein 516 variants with the human serum albumin binding capacity via site-specific fatty acid 517 conjugation. Sci. Rep. 7, 18041, 2017).

Precultured transformants were inoculated into a 2X·YT medium containing 100 μg/mL ampicillin (Sigma, #A0166) and incubated at 37° C. When the optical density at 600 nm (OD₆₀₀) reached 0.5, 1 mM isopropyl β-D-1-thiogalactopyranoside (IPTG, Thermo Fisher Scientific, #R0392) was added for Uox induction, and the mixture was incubated for 5 hours, and then the cells were pelleted by centrifugation at 5,000 g for 10 minutes. The cell pellets were stored at −80° C. until needed for use. In order to purify the Uox, the cell pellets were resuspended in lysis buffer (pH 7.4) containing 10 mM imidazole. The resuspended cell pellets were sonicated for 1 hour. After centrifugation at 12,000 rpm for 30 minutes, the supernatant was incubated with nickel-nitrilotriacetic acid (Ni-NTA) agarose beads (Qiagen, #30210) at 15° C. at 220 rpm for 1 hour. Then, the lysate incubated with the Ni-NTA agarose beads was poured into a polypropylene column (Qiagen) and washed thoroughly with washing buffer (pH 7.4) containing 20 mM imidazole.

Then, the purified Uox was eluted with elution buffer (pH 7.4) containing 250 mM imidazole, and the buffer was exchanged with PBS buffer (pH 7.4) using a PD-10 column (GE Healthcare Life Sciences). Finally, the purified Uox was concentrated to an appropriate concentration with a Vivaspin column (molecular weight cutoff [MWCO]: 10 kDa, Sartorius Corporation) according to the supplier's manual and stored at 4° C. until needed for use.

The molar extinction coefficient at 280 nm for Uox was calculated to be 53.520 M⁻¹cm⁻¹, and this was calculated by the following equation: (ε₂₈₀=(5,500×n_{Trp})+(1.490×n_{Tyr})+(125×n_{disulfide bond})).

Then, the concentration of Uox was determined using the Beer-Lambert law.

### EXPERIMENTAL EXAMPLE 2

### Formation of Uox-Palmitic Acid (PA) Conjugates According to Linker Length

In order to synthesize PA containing a DBCO group, 180 μM DBCO-amine (Click Chemistry Tools, #A103) and DBCO-PEG₄-amine (Click Chemistry Tools, #A103P) were each reacted with 900 μM NHS-PA (Sigma) at 37° C. for 20 hours, and thereby DBCO-PA and DBCO-PEG₄-PA were generated, respectively. The unreacted NHS groups of NHS-PA were quenched with an excess amount of Tris base (pH 7.4).

Uox-PA conjugates containing linkers with various lengths (UP01, UP02, UP03, and UP04) were prepared using FAs containing a reactive group (NHS-PA, DBCO-PA, and DBCO-PEG₄-PA).

First, 50 μM Uox and 500 μM NHS-PA were reacted in 20 mM sodium phosphate/0.1 M NaCl containing 0.30% (w/v) DCA at room temperature for 3 hours, and thereby UP01 was prepared.

Second, 50 μM Uox and 1,500 μM azidoacetic acid NHS ester (Click Chemistry Tools, #1070) were reacted in 20 mM sodium phosphate/0.1 M NaCl on ice for 2 hours and quenched with an excess amount of Tris base (pH 7.4), and thereby a Uox-azide intermediate (indicated as UA) was prepared. After desalting by Vivaspin (MWCO: 10 kDa), 50 μM UA was reacted with 100 μM DBCO-PA in 20 mM sodium phosphate/0.1 M NaCl containing 0.15% (w/v) DCA at room temperature for 3 hours, and thereby UP02 was prepared.

Third, 50 μM Uox and 1,500 μM azido-PEG₄-NHS ester (Click Chemistry Tools, #AZ103) were reacted in 20 mM sodium phosphate/0.1 M NaCl on ice for 2 hours and quenched with an excess amount of Tris base (pH 7.4), and thereby a Uox-PEG₄-azide intermediate (indicated as U4A) was prepared. After desalting by Vivaspin (MWCO: 10 kDa), 50 μM U4A was reacted with 100 μM DBCO-PA in 20 mM sodium phosphate/0.1 M NaCl containing 0.15% (w/v) DCA at room temperature for 3 hours, and thereby UP03 was prepared.

Fourth, 50 μM U4A was reacted with 100 μM DBCO-PEG₄-PA in 20 mM sodium phosphate/0.1 M NaCl containing 0.15% (w/v) DCA at room temperature for 3 hours, and thereby UP04 was prepared. Finally, for the Uox-PA conjugates, the buffer was exchanged with PBS buffer (pH 7.4) using a PD-10 column, and the Uox-PA conjugates were stored at 4° C. until needed for use.

### EXPERIMENTAL EXAMPLE 3

### Measurement of Concentration of Uox-PA Conjugates

The concentration of Uox-PA conjugates was measured by an enzyme-linked immunosorbent assay (ELISA) targeting a 6× His tag of Uox. 96-well microplates were coated with 100 μL of Uox standard or Uox-PA conjugates in PBS buffer at 4° C. overnight. In order to block non-specific binding, 5% (w/v) skim milk in PBS-T buffer (PBS containing 0.05% (v/v) Tween-20) was applied to the coated plates at room temperature for 2 hours, and the mixture was incubated with anti-6× His tag antibodies (Cell Signaling Technology [CST], #2365, at 1:1,000) for 2 hours.

After washing with PBS-T buffer, horseradish peroxidase (HRP)-conjugated anti-rabbit IgG (Cell Signaling Technology [CST], #7074, at 1:2,000) was applied to the plates for 1 hour. After washing with PBS-T buffer, a 3,3′,5,5′-tetramethylbenzidine (TMB, Sigma, #T4444) substrate was added for color development. The reaction was stopped with 1 M sulfuric acid. The absorbance at 450 nm was measured using a Synergy H1 multimode microplate reader (BioTek).

### EXPERIMENTAL EXAMPLE 4

### MALDI-TOF for Analysis of Uox and Uox-PA Conjugates

For the analysis of intact mass, the Uox and Uox-PA conjugates were desalted on a ZipTip C18 (Millipore Corporation) according to the manufacturer's protocol. A first layer was prepared by adding absolute ethanol, in which sinapinic acid (Sigma, #D7927) was dissolved, to a polished steel plate. The desalted Uox or Uox-PA conjugates were mixed with 1:1 of TA30 in which sinapinic acid was dissolved and then applied to make a secondary layer, then subjected to 400 mass analysis via microflex MALDI-TOF (Bruker Daltonics).

The mass analysis for each of the Uox and the Uox-PA conjugates was performed using flexControl-autoflex TOF/TOF software (Bruker Daltonics). The mass analysis was performed in a linear positive mode within a mass range from 20,000 Da to 50,000 Da. The MALDI-TOF-MS was calibrated using a Protein Standard II (20 kDa to 90 kDa; Bruker Daltonics) before the measurement according to the manufacturer's instructions.

A mass list with intensities and areas was derived manually (in the cases of Uox and UP01 of masses of major peaks) or automatically (in the cases of UP02, UP03, and UP04) using the flexAnalysis software (Bruker Daltonics).

The average mass of UP02, UP03, and UP04 was calculated by multiplying each area and mass of all peaks and then dividing its average value by the average area. The average number of conjugated PA of UP01 was obtained by multiplying the number of conjugated PA of the peak with the ratio of corresponding peak area to the total peak area. The average number of conjugated PA in UP02, UP03, and UP04 was obtained by taking into account the molecular weight of linker and PA in each average mass shift from that of Uox.

### EXPERIMENTAL EXAMPLE 5

### In Vitro Serum Albumin (SA) Binding Assay

Amine-binding plates (Thermo Fisher Scientific, #15110) were coated with 100 μL of MSA (10 μg/mL, Equitech-Bio Inc, #MSA62) or HSA (10 μg/mL, Sigma, #A3782) in PBS (pH 7.4) at 4° C. overnight. In order to block non-specific binding, 5% (w/v) skim milk in PBS-T buffer (pH 7.4) was added at room temperature for 2 hours. Uox and Uox-PA conjugates were prepared in PBS (pH 7.4) at predetermined concentrations (1.95 μg/mL to 1,000 μg/mL). Uox and Uox-PA conjugates in an amount of 50 μL were each incubated at room temperature for 2 hours, and then incubated with anti-6× His antibodies for 2 hours. After washing, HRP-conjugated anti-rabbit IgG was added thereto for 1 hour, a substrate was added thereto, and the reaction was stopped with 1 M sulfuric acid. The absorbance at 450 nm was detected with a Synergy H1 multimode microplate reader. The sigmoidal graph of OD₄₅₀ vs. concentration data was fitted to a Boltzmann equation using OriginPro 2018. The BC50 was defined as the concentrations of the Uox that bound 50% of a maximum amount bound to SA

### EXPERIMENTAL EXAMPLE 6

### In Vitro Uox Activity Assay

The Uox activity was measured by uric acid degradation. Specifically, the Uox activity was measured so that 50 nM of Uox or Uox-PA conjugates could be incubated with 100 μM uric acid (Sigma, #U2625) in 200 μL Uox assay buffer, which contained 50 mM sodium borate (pH 9.5) and 0.2 M NaCl. The Uox serum activity was measured by monitoring its OD at 293 nm. The molar absorptivity of uric acid at 293 nm is 12,300 M⁻¹cm⁻¹. In order to obtain the serum activity of Uox in the blood sample, 10 μL of serum was mixed with 190 μL of the assay buffer containing 100 μM uric acid, and then the mixture was monitored as described above. The serum activity of Uox was obtained in an arbitrary unit (mU/mL), in which one unit (mU) was defined as the amount of an enzyme that is used to catalyze the oxidation of 1 nmol of uric acid per minute at room temperature.

### EXPERIMENTAL EXAMPLE 7

### Measurement of Serum Half-Life in Mice

Uox activities of Uox and Uox-PA conjugates in vivo were examined by injecting 29 μM (1 mg/mL, based on Uox subunits) of each protein in 200 μL PBS (Thermo Fisher Scientific, #70011044) into the tail veins of 9-week-old female BALB/c mice (n=5).

Mice experiments were performed according to the guidelines of the Animal Care and Use Committee of the Gwangju Institute of Science and Technology (GIST). Blood samples (70 μL or less) were collected at 0 (10 minutes), 1, 2, 4, 8, 12, and 24 hours after the injection of Uox or Uox-PA conjugates, and were allowed to clot at room temperature for 30 minutes. Then, the resultants were centrifuged at 2,000 rpm at 4° C. for 15 minutes, and each serum was separated from the blood. The separated sera were each stored at 4° C. until needed for use.

### EXPERIMENTAL EXAMPLE 8

### FcRn/SA/Uox-PA Tertiary Complex Formation Assay

Amine-binding plates were coated with 100 μL of human FcRn (10 μg/mL, ACRO Biosystems, #FCM-H5286) or mouse FcRn (10 μg/mL, ACRO Biosystems, #FCM-M52W2) in PBS (pH 6.0) at 4° C. overnight. In order to block non-specific binding, 5% (w/v) skim milk in PBS-T buffer (pH 6.0) was added at room temperature for 2 hours. 100 μL of each of MSA (1 mg/mL) or HSA (1 mg/mL) in PBS (pH 6.0) was added at room temperature for 2 hours. After washing, 50 μL of each of Uox (1 mg/mL) and Uox-PA conjugates (1 mg/mL) in PBS (pH 6.0) was incubated at room temperature for 2 hours, and then incubated with anti-6× His antibodies for 2 hours. After washing, HRP-conjugated anti-rabbit IgG was added thereto for 1 hour, a substrate was added thereto, and the reaction was stopped with 1 M sulfuric acid. The absorbance at 450 nm was measured with a Synergy H1 multimode microplate reader.

### EXPERIMENTAL EXAMPLE 9

### Statistics and Data Analysis

All of the t-tests were two-sided tests. Statistical significance and individual tests are described in the figure legends.

### EXAMPLE 1

### Preparation of Uox-Palmitic Acid (PA) Conjugates

In order to examine the effect of linker length between FAs and therapeutic proteins on the increase of serum half-life, Uox-PA conjugates were prepared by the method of Experimental Example 2. First, rasburicase, which is a recombinant Uox derived from Aspergillus flavus, was obtained by overexpressing it in E. coli cells as previously reported (Hahn, I. Kwon, Generation of therapeutic protein 516 variants with the human serum albumin binding capacity via site-specific fatty acid 517 conjugation. Sci. Rep. 7, 18041, 2017). Then, the recombinant Uox expressed in E. coli cells was purified using its 6× His tag by metal affinity chromatography as previously reported.

It was confirmed that the purity of Uox analyzed by protein gel analysis was higher than 95% (FIG. 8). Additionally, since Uox is a homotetramer, a band for Uox subunit (35 kDa) was observed in a protein gel.

Then, in order to generate Uox-PA conjugates with various linker lengths, linker lengths were measured by analyzing the structures of serum albumin (SA), FcRn, Uox of therapeutic proteins for gout, and conjugates thereof. As a result, it was found that Uox-PA conjugates did not show a substantial increase in serum half-life compared to unmodified Uox (FIG. 1C). From the above result, it was possible to predict that Uox-PA substantially competes with the binding of FcRn to SA (FIG. 1C). Uox is a spherical protein, and the diameter of Uox was measured to be about 7 nm (FIG. 7B) based on the crystal structure (PDB ID: 1WS2).

In order to avoid the interference of the binding of FcRn to SA, the critical linker length between Uox and PA was prepared to be in a range of about 1 nm to 3 nm. In addition, by conjugating PA to Uox using N-hydroxysuccinimide (NHS)-amine and strain-promoted azide-alkyne cycloaddition (SPAAC) reactions, four Uox-PA conjugates with linker lengths in the range of 1 nm to 3 nm were prepared: UP01 (with a linker length of 0.25 nm); UP02 (with a linker length of 1.5 nm); UP03 (with a linker length of 2.8 nm); and UP04 (with a linker length of 4.8 nm) (FIG. 2A, and FIGS. 9A and 9B).

In the case of UP01, the conjugate was prepared by directly conjugating palmitic acid NHS ester (NHS-PA, FIG. 9C) to lysine residues of Uox through NHS-amine reactions, such that the distance between the ε-carbon in a lysine residue of Uox and a carbonyl carbon of PA could be about 0.25 nm, based on the estimation by Chem3D.

In order to increase the distance between Uox (i.e., the target protein) and PA, dibenzocyclooctyne (DBCO)-amine (FIG. 9D) or DBCO-PEG₄-amine (FIG. 9E) was reacted with NHS-PA, and thereby DBCO-PA or DBCO-PEG₄-PA was prepared, respectively (FIGS. 9F and 9G).

In the case of UP02, the conjugate was prepared as follows. Azidoacetic acid NHS ester was reacted with lysine residues of Uox (FIG. 9H). Then, the intermediate generated by the above reaction was reacted with DBCO-PA to prepare UP02, in which the distance between the ε-carbon in a lysine residue of Uox and a carbonyl carbon of PA was about 1.5 nm. Then, azide-PEG₄-NHS (FIG. 9I) was reacted with Uox, and then reacted with DBCO-PA or DBCO-PEG₄-PA, and thereby UP03 or UP04 was prepared, in which the distance between the ε-carbon in a lysine residue of Uox and a carbonyl carbon of PA was about 2.8 nm or about 4.8 nm, respectively.

The FA conjugation of the four Uox-PA conjugates was confirmed by protein gel analysis and mass spectrometric analysis. In a protein gel, the bands of the Uox-PA conjugates (UP01, 02, 03, and 04) were up-shifted from the band of unmodified Uox, thus confirming that the Uox was successfully modified (FIG. 2B and FIG. 10).

Additionally, it was confirmed that the bands of UP03 and UP04 with higher molecular weights were further up-shifted compared to those of UP01 and UP02 with lower molecular weights.

Since protein gel analysis only provides qualitative evidence of PA conjugation to Uox, for more quantitative analysis, matrix-assisted laser desorption ionization/time-of-flight (MALDI-TOF) mass spectrometry on Uox-PA conjugates as well as intact Uox were performed by the method of Experimental Example 4 so as to estimate the number of PAs conjugated to Uox (FIG. 11).

The mass of the intact monomeric Uox was measured to be 34,926 m/z (34,925 Da) experimentally, and it was confirmed that the value was consistent with its theoretical mass (34,930 Da).

In the case of UP01, NHS-PAs were directly conjugated to Uox, and thereby each major peak was assigned to Uox-PA conjugates with various numbers of PAs (PA 0 to PA 8).

In the case of UP01, the average number of PAs on each Uox subunit was 2.5, and in the cases of UP02, UP03, and UP04, it was difficult to assign each peak to a corresponding conjugate due to the combined characteristics of the numbers of linker intermediates and PAs conjugated to Uox.

Therefore, in order to estimate an average number of PAs, the average mass of each conjugate was used. The average masses of UP02, UP03, and UP04 were 36,328 Da, 37,733 Da, and 38,083 Da, respectively, thus indicating that the average numbers of PAs conjugated to each Uox subunit were 1.4, 2.1, and 1.9, respectively. Since Uox is a homo-tetramer, it was confirmed that a single molecule of UP01, UP02, UP03, and UP04 has about 10, 5.4, 8.4, or 7.6 PAs, respectively.

From the above results, it was confirmed that the number of PAs conjugated to a Uox single molecule, which is a homo-tetramer, was in a range of 6 to 10.

### EXAMPLE 2

### Examination of Binding Affinities of Uox-PA Conjugates to SA

In order to examine binding affinities of Uox-PA conjugates to serum albumin, considering that the half-lives of conjugates are measured in mice, binding affinities of Uox-PA conjugates to mouse serum albumin (MSA) were first examined by the method of Experimental Example 5. For potential clinical applications, the binding affinities of Uox-PA conjugates to SA were also examined Each well in a 96-well plate was coated with an appropriate amount of mouse serum albumin (MSA) or human serum albumin (HSA), and then Uox, UP01, UP02, UP03, or UP04 samples with various concentrations were incubated. After washing, the amount of Uox or Uox-PA bound to SA was measured by ELISA (FIG. 12A).

As a result, it was confirmed that as the concentrations of Uox-PA conjugates increased, the amount of Uox-PA increased but reached a plateau, which indicated that all of the four Uox-PA conjugates could bind to MSA and HSA (FIGS. 3A-3B).

Meanwhile, although the concentration of Uox increased, the amount of Uox did not increase as much as Uox-PA conjugates, thus indicating that the binding affinity of Uox to MSA and HSA deteriorates in the absence of PA.

A nonlinear curve fitting of a Boltzmann equation for these data enabled to obtain a half-maximal binding concentration (BC₅₀), which is a concentration of a Uox-PA conjugate at which the binding is reduced by half of the maximum binding.

In the case of MSA, the BC₅₀ values of UP01, UP02, UP03, and UP04 were 8.1 μM, 12.6 μM, 9.5 μM, and 13.2 μM, respectively (FIG. 3A). Additionally, in the case of HSA, the BC₅₀ values of UP01, UP02, UP03, and UP04 were 6.8 μM, 13.9 μM, 8.9 μM, and 13.3 μM, respectively (FIG. 3B).

The trend in binding affinities of Uox-PA conjugates to HSA, due to the above results, was similar to that to MSA, thus suggesting that Uox-PA conjugates bind to MSA and HSA in a similar manner.

Additionally, for both MSA and HSA, the BC₅₀ values between all of the four Uox-PA conjugates were different (i.e., less than a 2-fold difference), thus confirming that they have levels equivalent to those of SA binding affinities. Moreover, when BC₅₀ vs. linker length was plotted, no significant correlation was found (FIGS. 13A-13B).

Therefore, from the above experimental results, it was confirmed that the linker length did not have a direct impact on SA binding affinity. Additionally, the relatively small differences in BC₅₀ values between the Uox-PA conjugates may be due to the number of PAs conjugated to Uox, but it was not further analyzed because the number of PAs was not evaluated.

### EXAMPLE 3

### Measurement of Uric Acid Degradation Activity of Uox-PA Conjugates

In order to examine whether the PA conjugation to Uox has an effect on the enzymatic activity of uric acid degradation, the uric acid degradation activities of Uox-PA conjugates were measured by the method of Experimental Example 6.

The degradation rate of uric acid in the presence of each of the Uox-PA conjugates (UP01, UP02, UP03, and UP04) as well as Uox was measured by monitoring the changes in absorbance of uric acid at 293 nm.

As a result, the enzymatic activities of UP02, UP03, and UP04 were shown to be at a level comparable to that of Uox, but the enzymatic activity of UP01 was shown to be 40% lower compared to that of Uox (FIG. 14).

The significant reduction in the enzymatic activity of UP01 was thought to be due to the use of a higher concentration of DCA for a PA derivative with a low solubility during the conjugation reaction. Therefore, in the case of UP01, NHS-PA was directly conjugated to Uox. Although 0.15% of DCA was sufficient to prepare the other Uox-PA conjugates (UP02, UP03, and UP04), 0.15% of DCA was not sufficient for efficient conjugation of highly-hydrophobic NHS-PA, thus resulting in only a conjugation of 0.5 PA per Uox subunit (FIG. 11).

Accordingly, the DCA concentration was increased to 0.30% so as to prepare UP01 with PA conjugation, which corresponds to that of UP02, UP03, and UP04. In previous studies, it had been confirmed that DCA concentration greater than 0.15% can cause a loss in the enzymatic activity of Uox. However, the present inventors determined that the relatively low enzymatic activity of UP01 would not cause a problem in the measurement of serum half-life in vivo, because the remaining activities of the Uox-PA conjugates will be compared to the initial activities of the Uox-PA conjugates, which were injected to determine serum half-life in vivo.

That is, from the above results, it was confirmed that the uric acid degradation activities were not significantly changed because Uox formed conjugates with PAs.

### EXAMPLE 4

### Effect of Linker Length Between Uox and PA on Serum Half-Life

In order to evaluate the effect of linker length between Uox and PA on serum half-life, each single dose of Uox, UP01, UP02, UP03, and UP04 was intravenously injected into mice (n=5). Enzymatic activities of the serum samples obtained at set time point were analyzed. The logarithmic value of enzymatic activity value vs. time was fitted to a mono-exponential decay model, and the serum half-life was calculated by the method of Experimental Example 7 (FIG. 4A).

As a result, Uox was rapidly removed and showed serum half-life of 1.2 hours. As expected, it was confirmed that Uox-PA conjugates were removed more slowly than Uox. Specifically, the serum half-lives of UP01, UP02, UP03, and UP04 were 2.6, 5.2, 9.0, and 9.2 hours, respectively, which were significantly longer than that of Uox.

In particular, it was confirmed that when the linker length was in a range of 0.25 nm to 2.8 nm (i.e., UP01, UP02, and UP03), the half-life increased by about 2.1- to 7.5-fold, and this confirmed that the half-life was increased in direct proportion with the increase of the linker length (FIGS. 4A-4B).

Additionally, it was confirmed that when the linker length was in a range of longer than 2.8 nm and equal to or less than 4.8 nm, the increase rate of half-life in vivo was reduced, and thus, was maintained for 8 to 10 hours (FIG. 4B).

Separately, in order to examine critical factors which have an effect on the serum half-lives of Uox-PA conjugates, first, it was examined whether there is a correlation between an increase of half-life and the binding affinity of the Uox-PA conjugates to MSA or HSA.

As a result, when serum half-life vs. BC₅₀ of MSA or HSA was plotted, the coefficient of determination (indicated as R²) was 0.38 and 0.39, respectively, and it was confirmed that there was no meaningful correlation (FIGS. 15A-15B). This result was not surprising, considering the results of Example 2, where it was confirmed that linker length and SA binding in vitro were not correlated.

Then, it was examined whether the increase of half-life of Uox-PA conjugates was correlated to their linker lengths. As a result, the graph, which represented half-life vs. linker length, showed that the half-life increased as the linker length was increased up to 2.8 nm (FIG. 4B). The R² was 0.99, which suggests that there is a very strong correlation.

In contrast, when the linker length was increased from 2.8 nm to 4.8 nm, it did not significantly change serum half-life in vivo.

That is, from the above results, it was confirmed that the distance between PA and Uox has a critical role in the extension of serum half-life in vivo. In particular, it was confirmed that when the linker length of the Uox-PA conjugate is 3 nm or less, it has an effect of increasing the half-life.

### EXAMPLE 5

### Formation of Tertiary Structure of FcRn/SA/Uox-PA Conjugate which is Dependent on Linker Length

In order to confirm that the competition of Uox-PA conjugates with the binding of FcRn to SA depends on the linker length, whether the increase of serum half-life correlates with the formation of an FcRn/SA/Uox-PA tertiary structure was examined by the method of Experimental Example 8.

As a control, the binding of Uox-PA conjugates to MSA or HSA at pH 6.0 was analyzed.

As a result, all of the four Uox-PA conjugates showed a significantly improved binding to MSA or HSA compared to Uox (FIGS. 5A and 5C).

Additionally, there was no significant difference between the four Uox-PA conjugates in their binding to SA (FIGS. 5A and 5C), which was consistent with in vitro SA binding assay results at pH 7.4 (FIGS. 3A-3B). From the above results, it was reconfirmed that Uox-PA conjugates had equivalent SA binding abilities.

Then, the formation of an FcRn/SA/Uox-PA tertiary structure was examined by measuring the amount of Uox-PA binding to an FcRn/SA complex in 96-well plates, as illustrated in FIG. 12B.

As a result, as the linker length increased from UP01 to UP03, the amount of the FcRn/SA/Uox-PA tertiary structure formed increased (FIGS. 5B and 5D). In addition, similarly to the increase of serum half-life, the tertiary complex formation of UP04 was not significantly different from that of UP03 (FIGS. 5B and 5D).

Additionally, it was confirmed that when the serum half-life vs. the amount of the FcRn/SA/Uox-PA tertiary structure was plotted, the correlation was very strong (R²=0.99 (FIGS. 16A-16B)). The very strong correlation indicates that the increase of half-lives of Uox-PA conjugates is dependent on the successful formation of FcRn/SA/Uox-PA tertiary complexes.

That is, the above results indicate that FA conjugation was applied to large therapeutic proteins by introducing linkers with suitable lengths so as to extend their half-lives. These results provide a better understanding with respect to the mechanism of half-life extension of FA-conjugated proteins and suggest the direction of the method of FA conjugation for large proteins, and thus can contribute to the development of next-generation FA-conjugated drugs with more diverse and complex properties.

From the foregoing, one of ordinary skill in the art to which the present invention pertains will be able to understand that the present invention may be embodied in other specific forms without modifying the technical concepts or essential characteristics of the present invention. In this regard, the exemplary embodiments disclosed herein are only for illustrative purposes and should not be construed as limiting the scope of the present invention. On the contrary, the present invention is intended to cover not only the exemplary embodiments but also various alternatives, modifications, equivalents, and other embodiments that may be included within the spirit and scope of the present invention as defined by the appended claims.

1. A conjugate of General Formula 1 below capable of extending in vivo half-life according to the length from Uox to Y: [chemical structure]
wherein in General Formula 1 above,
Uox is urate oxidase;
X is a polymer; and
Y is a fatty acid.

2. The conjugate of claim 1, wherein the in vivo half-life of the conjugate increases when the length from Uox to Y is in a range between greater than 0.2 nm and 3 nm or less.

3. The conjugate of claim 1, wherein the in vivo half-life of the conjugate is maintained for 8 to 10 hours when the length from Uox to Y is in a range between greater than 3 nm and 5 nm or less.

4. The conjugate of claim 1, wherein the Uox has a tetrameric structure.

5. The conjugate of claim 1, wherein the polymer comprises polyethylene glycol (PEG), dibenzocyclooctyne (DBCO), or a combination thereof.

6. The conjugate of claim 1, wherein the fatty acid is a C₁₀₋₂₀ fatty acid.

7. The conjugate of claim 6, wherein the fatty acid is one or more selected from the group consisting of palmitic acid (PA), lauric acid, myristic acid, and stearic acid.

8. The conjugate of claim 1, wherein the conjugate is one or more selected from the group consisting of Formula 1 to Formula 4 below:

[chemical structure]

9. The conjugate of claim 1, wherein the conjugate forms a complex in vivo with serum albumin (SA) and neonatal Fc receptor (FcRn).

10. A pharmaceutical composition with increased in vivo half-life for preventing or treating gout, comprising the conjugate of claim 1 or a pharmaceutically acceptable salt thereof.

11. A method for preventing or treating gout, comprising a step of administering the conjugate of General Formula 1 below capable of extending in vivo half-life according to the length from Uox to Y; or a pharmaceutically acceptable salt thereof to a subject. [chemical structure]
wherein in General Formula 1 above,
Uox is urate oxidase;
X is a polymer; and
Y is a fatty acid.

12. The method of claim 11, wherein the in vivo half-life of the conjugate increases when the length from Uox to Y is in a range between greater than 0.2 nm and 3 nm or less.

13. The method of claim 11, wherein the in vivo half-life of the conjugate is maintained for 8 to 10 hours when the length from Uox to Y is in a range between greater than 3 nm and 5 nm or less.

14. The method of claim 11, wherein the polymer comprises polyethylene glycol (PEG), dibenzocyclooctyne (DBCO), or a combination thereof.

15. The method of claim 11, wherein the fatty acid is a C₁₀₋₂₀ fatty acid.

16. The method of claim 11, wherein the fatty acid is one or more selected from the group consisting of palmitic acid (PA), lauric acid, myristic acid, and stearic acid.

17. The method of claim 11, wherein the conjugate is one or more selected from the group consisting of Formula 1 to Formula 4 below: [chemical structure]
